(19) United States
(12) Patent Application Publication
(10) Pub. No.: US 2025/0132003 A1
(43) Pub. Date: Apr. 24, 2025

(54) SimPillRx

(71) Applicant: Barbara Butkiewicz, Philadelphia, PA (US)

(72) Inventor: Barbara Butkiewicz, Philadelphia, PA (US)

(21) Appl. No.: 18/491,759

(22) Filed: Oct. 21, 2023

(51) Int. Cl. G16H 20/10; G06K 19/06; G16H 15/00

(52) U.S. Cl. CPC G16H 20/10; G16H 15/00; G06K 19/06037

(57) ABSTRACT

A system which addresses the challenge of limited resources for medical professionals to truncate each and every medicine in real time. Instead of relying on QR codes or vague recommendations, patients can receive much fewer pages of their medications truncated access at a centralized platform from a system that offers organized and prioritized information. The tool provides a truncated and full of information selection for each and every prescribed medicine, allowing patients to focus on the most critical aspects of their medications. This ensures a more efficient learning process and empowers patients to become knowledgeable about their medications all at once.

### FIELD OF THE INVENTION

The invention relates to methods and apparatus for condensing medication information.

### BACKGROUND OF THE INVENTION

Effective medication communication directly links to improved medication adherence, better health outcomes, decreased hospitalizations and increased patient satisfaction. The high prevalence of polypharmacy is problematic for patients and impacts retention of medication knowledge. Nurses and patients alike complain about the overabundance of information provided regarding medications. Speaking of overabundance of information regarding medications, oftentimes, patients use a QR code to access medical information about a single drug. A QR code (Quick-Response Code) is well known and heavily used today. A QR code is a small coded patch resembling a 2 dimensional bar code (but different) that is designed to be viewed by the camera or image sensor in a cellular smartphone, pad or other portable device. Typically the QR code simply returns a URL that the user browser then moves to.

Typically, when providing education to patients, nurses use an online platform, Krames, to print medication sheets which average 1-3 pages per medication. On an oncology unit, patients regularly have 15 medications ordered. Fifteen medications equates to 45 pages of information. A diabetic patient admitted with ketoacidosis can be prescribed 15 medications also. When a patient is admitted with COPD (Chronic Obstructive Pulmonary Disease), they typically are prescribed 20 medications, resulting in 60 pages of information. A patient admitted for congestive heart failure and who also has leukemia must be prescribed 33 medications, resulting in 99 pages of information.!!! Something must be done.

Another reason this preferred embodiment is necessary is because hospital reimbursement from the Centers for Medicare & Medicaid Centers is linked directly to the HCAHPS (Hospital Consumer Assessment and Healthcare Providers and Systems) scores.

Communication about Medication is a domain on the HCAHPS Survey. The HCAHPS Survey is a survey that patients receive after discharge to rate their hospital experience.

The questions the nurses ask are: 1. Did the nurse explain why you were taking the medications? 2. Did the nurse explain the possible side effects of the medications.

### SUMMARY OF THE INVENTION

SimPillRx is a website that solves the problem of information overload. With this unique and distinct website, SimPillRx, instead of 45 pages for fifteen medications, now there is 3 pages. Instead of 60 pages of information, now there are 5 pages. This website condenses pages and pages of medicinal information and reduces it to summarized descriptions of each and every medication. This condensation is as follows. The website SimPillRx lists 1. each medication 2. indications for use, 3. several common side effects, and 4. a QR Code that links to an online platform which displays the entire description of the medication. The feedback from nurses and patients is positive. The form is clean, concise, easily readable, and understandable. Patients can reference it easily and take it home to hang on their refrigerator.

It is an object of this invention SimPillRx, that revolutionizes medicinal handouts and immensely improves the information the patients can not only learn about the medicine but can help patients understand the uses and limitations and potential side effects of these medications.

One of the core features of SimPillRx is its ability to provide instant access to crucial information, eliminating confusion and over information. The tool ensures that medical professionals and their patients have all the necessary information at their fingertips, starting from the initial conversation about what is wrong to the completion of the diagnosis and medicines recommended.

Embodiments of this invention relate to methods and apparatus for narrowing down the amount of medicinal information the patient should be taking to facilitate the dosage schedule, while keeping in mind, the big picture, of the patients diagnosis. It is simply a website, controlled by a medical professional input, which condenses the medical information for any given medicine.

### BRIEF DESCRIPTIONS OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an example of a page of condensed medicines, according to certain embodiments.

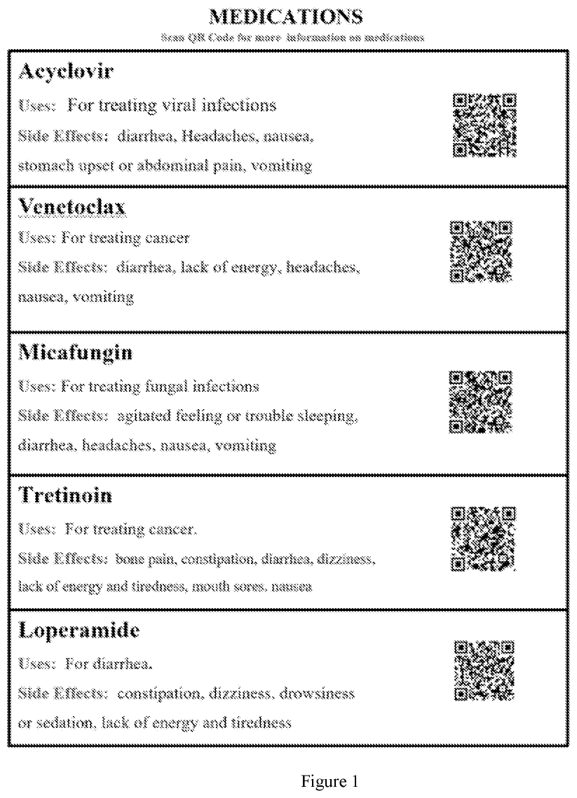

### DETAILED DESCRIPTION OF THE INVENTION

In preferred embodiments of this invention, medical professionals use a website, SimPillRx, to produce condensed medical information for each and every medicine prescribed for the patient with a certain diagnosis.

In preferred embodiments of this invention the website, SimPillRx, lists each medication, indications for use, several common side effects, and a QR Code that links to an online platform which displays the entire description of the medication and more.

In preferred embodiments of this invention, the medical professional uses medicinal specific intelligent software, which based on a diagnosis of a patient, generates condensed medicinal information sheets

By leveraging SimPillRx, users gain access to a vast repository of knowledge and expertise related to medicine information truncating, such that each and every prescribed medicine lists each medication, indications for use, several common side effects, and a QR Code that links to an online platform which displays the entire description of the medication and more. This comprehensive information empowers patients to make informed decisions regarding their medications, saving valuable time for both patients and their medical professionals and potentially saving patients lives. SimPillRx is a comprehensive software tool designed to condense the information overload by providing succinct and truncated medicinal information to the patients. The tool addresses several pain points and challenges faced by medicinal professionals and patients alike and aims to streamline operations, enhance patient satisfaction, and maximize efficiency throughout the information transfer process.

Furthermore, SimPillRx addresses the issue of information overload. It serves as a virtual trainer, providing continuous guidance and acting as a reliable resource for patients. With this tool, medical professionals can ensure that their patients have the necessary expertise and information to take their medications correctly and timely. The value provided by SimPillRx is comparable to having a medical professional individually truncating each and every medicine in real time, potentially saving the time it would take to do and the potential disorientation of the patient upon receiving such truncation in real time.

Additionally, SimPillRx addresses the challenge of limited resources for medical professionals to truncate each and every medicine in real time. Instead of relying on QR codes or vague recommendations, patients can receive much fewer pages of their medications truncated access at a centralized platform, SimPillRx, that offers organized and prioritized information. The tool provides a truncated and full of information selection for each and every prescribed medicine, allowing patients to focus on the most critical aspects of their medications. This ensures a more efficient learning process and empowers patients to become knowledgeable about their medications all at once.

### Particular Applications to Computer Devices

The methods and apparatus applied to this invention may include a plurality of different computing device types. In general, a computing device type may be a computer system or computer server. The computing device may be described in the general context of computer system executable instructions, such as program modules, being executed by a computer system (described for example, below). In some embodiments, the computing device may be a cloud computing node (for example, in the role of a computer server) connected to a cloud computing network (not shown). The computing device may be practiced in distributed cloud computing environments where tasks are performed by remote processing devices that are linked through a communications network. In a distributed cloud computing environment, program modules may be located in both local and remote computer system storage media including memory storage devices.

The computing device may typically include a variety of computer system readable media. Such media could be chosen from any available media that is accessible by the computing device, including non-transitory, volatile and non-volatile media, removable and non-removable media. The system memory could include random access memory (RAM) and/or a cache memory. A storage system can be provided for reading from and writing to a non-removable, non-volatile magnetic media device. The system memory may include at least one program product having a set (e.g., at least one) of program modules that are configured to carry out the functions of embodiments of the invention. The program product/utility, having a set (at least one) of program modules, may be stored in the system memory. The program modules generally carry out the functions and/or methodologies of embodiments of the invention as described herein.

As will be appreciated by one skilled in the art, aspects of the disclosed invention may be embodied as a system, method or process, or computer program product. Accordingly, aspects of the disclosed invention may take the form of an entirely hardware embodiment, an entirely software embodiment (including firmware, resident software, micro-code, etc.) or an embodiment combining software and hardware aspects “system.” Furthermore, aspects of the disclosed invention may take the form of a computer program product embodied in one or more computer readable media having computer readable program code embodied thereon.

Aspects of the disclosed invention are described above with reference to methods, apparatus (systems) and computer program products according to embodiments of the invention. It will be understood that each block of the block diagrams, and combinations of blocks in the flowchart illustrations and/or block diagrams, can be implemented by computer program instructions. These computer program instructions may be provided to the processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

### Other Embodiments

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

1. A software program to provide patients with condensed medicinal paperwork.

2. The method of claim 1 wherein said medical professional uses a software program, SimPillRx, to produce condensed medicinal data sheets.

3. The method of claim 1 wherein the condensed medicinal information consists of lists each medication, indications for use, several common side effects, and a QR Code that links to an online platform which displays the entire description of the medication.

4. The method of claim 1, wherein the condensed medicinal paperwork is dependent upon those medications prescribed for a patient.

5. The method of claim 4, wherein those medicines prescribed for a patient depend upon a diagnosis of the patient.
